(19) United States
(12) Patent Application Publication
(10) Pub. No.: US 2025/0352703 A1
(43) Pub. Date: Nov. 20, 2025

(54) Piezoelectric Conductive Composite Stent And Preparation Method Thereof

(71) Applicant: Shanghai Sixth People's Hospital, Shanghai (CN)

(72) Inventors: Xiangyun YAO, Shanghai (CN); Cunyi FAN, Shanghai (CN); Lingchi KONG, Shanghai (CN)

(73) Assignee: Shanghai Sixth People's Hospital, Shanghai (CN)

(21) Appl. No.: 18/279,337

(22) PCT Filed: May 30, 2023
(86) PCT No.: PCT/CN2023/097236

§ 371 Date: Dec. 27, 2024

(30) Foreign Application Priority Data
May 11, 2022 (CN) 202210509892.2

(51) Int. Cl. A61L 31/14; A61L 27/08; A61L 27/18; A61L 27/56; A61L 31/02; A61L 31/06; H10N 30/05; H10N 30/077; H10N 30/092; H10N 30/098; H10N 30/85; H10N 30/857

(52) U.S. Cl. CPC A61L 31/146; A61L 27/08; A61L 27/18; A61L 27/56; A61L 31/024; A61L 31/06; H10N 30/05; H10N 30/077; H10N 30/092; H10N 30/098; H10N 30/852; H10N 30/857; A61L 2400/12; A61L 2430/32

(57) ABSTRACT

The present invention provides a piezoelectric conductive composite stent and a preparation method thereof, the length of the piezoelectric conductive composite stent is 1 cm-3 cm, the inner diameter of the piezoelectric conductive composite stent is 2.5 mm-3.5 mm, and the thickness of the pipe wall of the piezoelectric conductive composite stent is 0.4 mm-0.45 mm. The piezoelectric conductive composite stent comprises: an inner layer and an outer layer sleeved outside the inner layer, and a plurality of nano grooves are provided in the peripheral face of the outer layer; wherein, the inner layer is prepared from polycaprolactone dissolved in a binary organic solvent; the outer layer is prepared from at least one of the polycaprolactone dissolved in a binary organic solvent, polyvinylpyrrolidone dissolved in a binary organic solvent, nanoparticles of metal organic framework materials, or nanoparticles of graphene or its derivatives.

### BACKGROUND OF THE INVENTION

### 1. Field of the Invention

This present invention relates to the field of neural catheters, and more particularly, to a piezoelectric conductive composite stent and a preparation method thereof.

### 2. Description of the Related Art

Peripheral nerve defects are clinically high-prevalent, with high disability rates and difficult treatment. The development of tissue engineering products has provided a solution for long peripheral nerve defects. The construction of peripheral nerve regeneration microenvironment needs to meet the four major elements of immune balance, microvascularization, microelectrical conduction and metabolic homeostasis, and the lack of any of them will lead to the failure of nerve repair. By preparing multifunctional electroactive neural catheters with inflammatory regulatory effects, it is expected to reconstruct the peripheral nerve microenvironment and improve repair efficiency.

Macrophages are rapidly activated after peripheral nerve injury, and a large number of macrophages are recruited to remove cellular debris from the injured area, causing Schwann cell dedifferentiation and initiating tissue repair, which is of great significance for early neural regeneration. Peripheral nerves have good electrical activity, and restoring electrical signaling through electrical stimulation is the most direct and effective way to promote nerve regeneration after injury. In addition, it has been demonstrated that electrical stimulation or bioelectric signals can affect cell membrane potential, thereby regulating macrophage phenotypic polarization and functional transition, and participating in the re-establishment of the balance of the inflammatory microenvironment after injury. At present, there are some improved designs in neural catheter products at home and abroad, which have been proven to accelerate nerve regeneration by introducing various conductive materials with high biological safety. However, the generation and transmission of electrical signals cannot be realized by simply introducing conductive materials, and the fact that the current is interrupted after nerve injury cannot be changed, and external current stimulation is often required in practical applications. Exogenous electrical stimulation has unfavorable factors such as inconvenient operation and easy infection at the stimulation site, so it is of great significance to develop a self-generating neural catheter that does not require exogenous electrical stimulation.

Metal organic framework (MOF) is a kind of organic/inorganic hybrid materials with good piezoelectric properties and intramolecular pores, which are formed by self-assembly of organic ligands and metal ion agglomeration, and have the advantages of low density, high porosity, large specific surface area, adjustable pore size, surface modification ability and diversity of topology. Deformation can occur under the action of tiny machinery, and the relative displacement of positive and negative ions in the unit cell makes the positive and negative charge centers no longer coincide, resulting in macroscopic polarization of the crystal, and a different charge appears on both ends of the stent. The damaged tissue contains too many reactive oxygen species and acidic metabolites, which destroy the MOF crystal structure and produce deformation, so that metal ions in MOF particles, such as Cu²⁺ and Zn²⁺, are released in peripheral nerve tissues, and the slowly-released metal ions can directly act on the membrane potential of the cell membrane and regulate the ion channel state; therefore, unlike traditional piezoelectric materials, MOF particles can directly reverse the polarization direction of macrophages in regenerated tissues, thereby inhibiting excessive inflammation. In addition, the slow-release Cu²⁺ and Zn²⁺ can activate intracellular superoxide dismutase and protect cells from oxidative stress.

The existing functionalized electroactive neural catheters can provide accurate and efficient bioelectric stimulation to promote the proliferation and differentiation of Schwann cells, but the electrical response effect of the material-cell interface at the macrophage level is not obvious, and it is difficult to achieve immune remodeling in the peripheral nerve microenvironment with the help of bionic current. This is because the existing research has not deeply explored the biological effects and mechanism of neural ducts in the process of peripheral nerve repair, so it is impossible to regulate the ion channel state of immune cells and activate the immune metabolism cascade reaction with the help of nerve electrical activity to build a neural regeneration microenvironment.

### SUMMARY OF THE INVENTION

Object of the present invention is to provide a piezoelectric conductive composite stent and preparation method thereof for the deficiencies in the prior art.

In order to achieve the above object, the technical solution taken by the present invention is:

A first aspect of the present invention is provided with a piezoelectric conductive composite stent, the length of the piezoelectric conductive composite stent is 1 cm-3 cm, the inner diameter of the piezoelectric conductive composite stent is 2.5 mm-3.5 mm, and the thickness of the pipe wall of the piezoelectric conductive composite stent is 0.4 mm-0.45 mm; the piezoelectric conductive composite stent comprises: an inner layer and an outer layer sleeved outside the inner layer, and a plurality of nano grooves are provided in a peripheral face of the outer layer;
wherein the inner layer is prepared from polycaprolactone dissolved in a binary organic solvent;
the outer layer is prepared from at least one of a polycaprolactone dissolved in a binary organic solvent, polyvinylpyrrolidone dissolved in a binary organic solvent, nanoparticles of metal organic framework materials, or the nanoparticles of graphene or its derivatives.

Preferably, the metal organic framework material is UIO-66-NH₂.

Preferably, the graphene or its derivatives comprise: at least one of graphene, graphene oxide or reduced graphene oxide.

Preferably, the binary organic solvent is a dichloromethane/dimethylformamide organic solvent, the volume ratio of dichloromethane to the dimethylformamide is in the range of 2 to 4.

A second aspect of the present invention is provided with a method for preparing the piezoelectric conductive composite stent described above, comprising the steps of:
S1, adding polycaprolactone to a binary organic solvent, and after sonication, obtaining a spinning liquid of the inner layer; adding polycaprolactone and polyvinylpyrrolidone to binary organic solvents, and after sonication, adding nanoparticles of graphene or its derivatives and nanoparticles of metal organic framework materials, and after uniform treatment, obtaining a spinning liquid of the outer layer;
S2, electrostatic spinning the spinning liquid of the inner layer prepared by step S1 and the spinning liquid of the outer layer prepared by step S1 together, and after drying, washing several times, obtaining the piezoelectric conductive composite stent.

Preferably, in step S1, the temperature of the sonication is 10° C.-20° C., and the time of the sonication is 20 min-40 min.

Preferably, in step S1, the mass concentration of polycaprolactone in the spinning liquid of the inner layer is 15%-20%.

Preferably, in step S1, in the spinning liquid of the outer layer, the mass concentration of polycaprolactone is 8%-12%; the mass concentration of polyvinylpyrrolidone is 4%-8%; the mass concentration of nanoparticles of graphene or its derivatives is 1%-2%; the mass concentration of nanoparticles in metal organic framework materials is 1%-2%.

Preferably, in step S2, the electrostatic spinning comprises: adding, respectively, the of spinning liquid of the inner layer and the spinning liquid of the outer layer to two syringes, wherein the two syringes share a nozzle, the model of the nozzle is 19, a voltage of the spinning is 10 kV-20 kV, a receiving distance of the receiving rod is 18 cm-20 cm, and a speed of the push pump is 1.8 mL/h-2.5 mL/h; the mold speed for electrostatic spinning the inner layer is 10 rpm-20 rpm, and the mold speed for electrostatic spinning the outer layer is 70 rpm-90 rpm.

Preferably, in step S2, the detergent used for washing comprises: alcohol or/and water.

The present invention adopts the above technical solution, compared with the prior art, has the following technical effects:
(1) the present invention constructs a piezoelectric conductive composite stent by coaxial electrostatic spinning integrated forming technology, using UIO-66-NH2 nanoparticles to act as piezoelectric catalytic response materials, and using nanoparticles of graphene or its derivatives to act as conductive materials, under the mechanical force of ultrasonic waves, the piezoelectric crystals in the stent are deformed, and the generated self-generated electricity is conducted by conductive particles, promoting the transmission of electrical signals on the surface of the stent from the proximal end to the distal end, and guiding the elongation of the distal end of the regenerative nerve axon;
(2) the material has small toxic, side effects, and good biocompatibility, implantation of an external power supply or electrode is not needed, the tissue regeneration speed can be effectively improved, the pain and inconvenience of patients is reduced, the risk of infection is decreased;
(3) the piezoelectric conductive composite stent of the present invention can affect the bioelectricity level of macrophage cell membranes, reduce calcium ion influx and thereby reduce the expression of inflammatory signaling pathways, and at the same time promote utilization and productivity of glucose in cells by changing the macrophage metabolic mode, thereby regulating and controlling the polarization of macrophages from proinflammatory phenotype to anti-inflammatory phenotype;
(4) the piezoelectric conductive composite stent of the present invention combines with ultrasound-assisted therapy, therefore, it can promote axon regeneration and myelination, and has good clinical application prospects.

### BRIEF DESCRIPTION OF THE DRAWINGS

FIG. 1A shows transmission electron microscopy of UIO-66-NH₂ nanoparticles; FIG. 1B shows a high-resolution transmission electron microscopy of UIO-66-NH₂ nanoparticles.

FIG. 2A is a transmission electron microscopy diagram of particles of reduced graphene oxide; FIG. 2B is a high-resolution transmission electron microscopy of particles of reduced graphene oxide;

FIG. 3 is a real shot of a piezoelectric conductive composite stent in an embodiment of the present invention;

FIG. 4 is the scanning electron microscopy diagram of electrostatic spinning fiber;

FIG. 5 is a piezoelectric microscope photograph of a piezoelectric conductive composite stent in one embodiment of the present invention, in which FIG. 5A is a topography diagram, FIG. 5B is an amplitude image, FIG. 5C is a phase diagram;

FIG. 6 is immunofluorescence staining result diagram of the dorsal root ganglion neuron on the piezoelectric conductive composite stent and on the control group PCL duct in an embodiment of the present invention, in which FIG. 6A is the piezoelectric conductive composite stent group, FIG. 6B is the PCL group;

FIG. 7 is transmission electron microscopy detection results of the regenerative nerve in the piezoelectric conductive composite stent and in the control group PCL duct stent in an embodiment of the present invention, in which FIG. 7A is the piezoelectric conductive composite stent group, FIG. 7B is the PCL group;

FIG. 8 is the polarization phenotype change of a macrophage cultured on a piezoelectric conductive composite stent and on a control group PCL duct stent in an embodiment of the present invention; in which FIG. 8A-D show the expression level of proinflammatory phenotype (iNOS, TNF-α) gene for whole RNA extraction of RAW264.7 cells cultured on stents; FIG. 8E is the piezoelectric conductive composite stent group, and FIG. 8F is the PCL group;

FIG. 9 is the expression of CaMKII activation in cells and inflammatory factor NF-κB in the macrophages cultured on a piezoelectric conductive composite stent (Exp) and a control group PCL duct stent (Con), and the addition of ATP-gated potassium channel blocker glibenclamide (Gliben) and voltage-gated calcium blocker (Vera) in an embodiment of the present invention;

FIG. 10 is the expression of the glycolytic metabolism key rate-limiting enzyme, hexokinase (HK−1), 6-phosphate fructokinase (PFKM), pyruvate kinase (PKM1) and tricarboxylic acid cycle key rate-limiting enzyme, citrate synthase (CS), isocitrate dehydrogenase (IDH2), ketoglutarate dehydrogenase (OGDH) expression in the macrophages cultured on piezoelectric conductive composite stent and a control group PCL duct stent in an embodiment of the present invention;

FIG. 11 is the infiltration of the macrophages of regenerative nerve in the piezoelectric conductive composite stent and in the control group PCL duct in an embodiment of the present invention; in which FIG. 11A shows the iNOS staining results of piezoelectric conductive composite stent group, FIG. 11B shows the iNOS staining results of PCL group, FIG. 11C shows the CD206 staining results of piezoelectric conductive composite stent group, and FIG. 11D shows the CD206 staining results of PCL group.

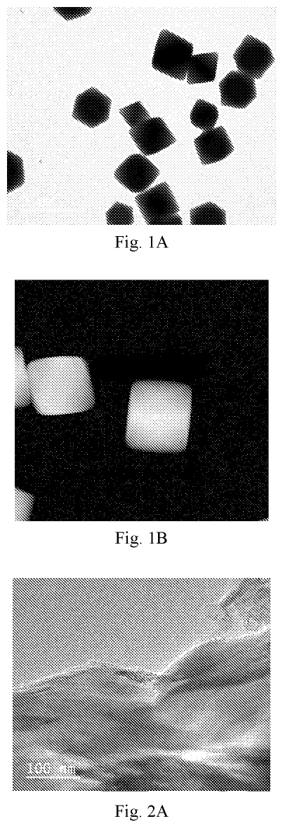

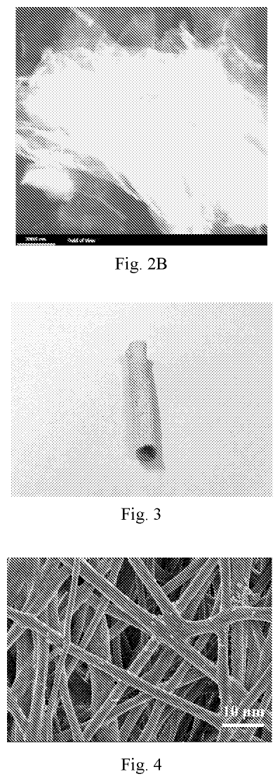

### DETAILED DESCRIPTION

The following will be combined with the drawings in the embodiments of the present invention, the technical solutions in the embodiments of the present invention are clearly and completely described, obviously, the described embodiments are only a part of the embodiments of the present invention, not all embodiments. Based on the embodiments in the present invention, all other embodiments obtained by those of ordinary skill in the art without performing creative labor, fall within the scope of the protection of the present invention.

It should be noted that, without conflict, embodiments in the present invention and the features in embodiments may be combined with each other.

The present invention is further described below in conjunction with the accompanying drawings and specific embodiments, but is not used as a limitation of the present invention.

### Example

In the example, there is provided a piezoelectric conductive composite stent preparation method, comprises the steps of:

S1, 1.8 g of polycaprolactone (PCL) (purchased from Sigma) to 10 mL of dichloromethane/dimethylformamide organic solvent (the volume ratio of dichloromethane to dimethylformamide is 3:1) (purchased from Shanghai Lingfeng Chemical Reagent Co., Ltd.) was added, and after dispersing for 30 min at 15° C., the spinning liquid of the inner layer was obtained; 1 g of PCL and 0.8 g of polyvinylpyrrolidone (PVP) (purchased from Aladdin) were added to 10 mL of dichloromethane/dimethylformamide organic solvent (the volume ratio of dichloromethane to dimethylformamide is 3:1), after ultrasonic dispersion at 15° C. for 30 min, nanoparticles of 1 wt %-2 wt % reduced graphene oxide and nanoparticles of 1 wt %-2 wt % UIO-66-NH2 were added, and after uniform shaking for 12 hours, the spinning liquid of the outer layer was obtained;

S2, the spinning liquid of the inner layer prepared in step S1 and the spinning liquid of the outer layer prepared in step S1 were respectively added to two 10 mL syringes, the two syringes shared a nozzle, the model of the nozzle was 19, the voltage of the spinning was 16 kV, the receiving distance of the receiving rod was 18 cm, the speed of the push pump was 2.5 mL/h, the negative pressure at both ends of the insulating rod was-1.5 kV, the spinning liquid of the inner layer was sprayed onto the mold with a speed of 15 rpm for 4 minutes, and the inner layer of orientation arrangement was obtained. The spinning liquid of the outer layer was sprayed onto the mold with a speed of 80 rpm for 40 minutes, and an outer layer with messy arrangement of fibers was obtained. After drying, washing 3 times with alcohol and water, removing PVP, and the groove structure was obtained on the surface of the fiber, i.e., the piezoelectric conductive composite stent was obtained.

### Control Example

The control example provides a method for preparing a PCL catheter stent, comprises the steps of:

S1, 1.8 g of polycaprolactone (PCL) was added to 10 mL of dichloromethane/dimethylformamide organic solvent (the volume ratio of dichloromethane to dimethylformamide is 3:1), stirring at room temperature for 8 h, i.e., the spinning liquid of the inner layer was obtained; 1 g PCL and 0.6 g of polyvinylpyrrolidone (PVP) was added to 10 mL of dichloromethane/dimethylformamide organic solvent (the volume ratio of dichloromethane to dimethylformamide is 3:1), stirring at room temperature for 8 h, i.e., the spinning liquid of the outer layer was obtained;

S2, spinning liquid of the inner layer prepared in step S1 and the spinning liquid of the outer layer were respectively added to two 10 mL syringes, the two syringes shared a nozzle, the model of the nozzle was 19, the voltage of the spinning was 16 kV, the receiving distance of the receiving rod was 18 cm, the speed of the push pump was 2.5 mL/h, the negative pressure at both ends of the insulating rod was-1.5 kV, the spinning liquid of the inner layer was sprayed onto the mold with a speed of 15 rpm for 4 minutes, and the inner layer of orientation arrangement was obtained. The spinning liquid of the outer layer was sprayed onto the mold with a speed of 80 rpm for 40 minutes to obtain the outer layer with messy arrangement of fibers. After drying, washing 3 times with alcohol and water, removing PVP, and the groove structure was obtained on the surface of the fiber, i.e., PCL duct stents was obtained.

### Detection Example

The appearance of the piezoelectric conductive composite stent prepared by the example was observed by naked eye, and the length, wall thickness and inner diameter were measured. The results were shown in FIG. 3. The length of the piezoelectric conductive composite stent was 1.5 cm, the inner diameter was 2.5 mm, and the wall thickness of the tube was 0.4 mm.

The piezoelectric conductive composite stent was observed under scanning electron microscope, and the results were shown in FIG. 4.

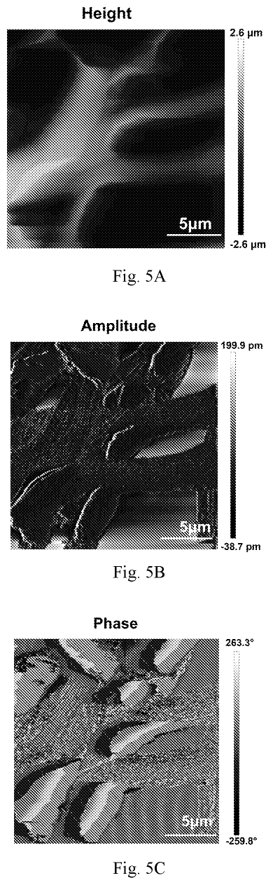

The piezoelectric conductive composite stent was observed under a piezoelectric force microscope, and the results were shown in FIG. 5.

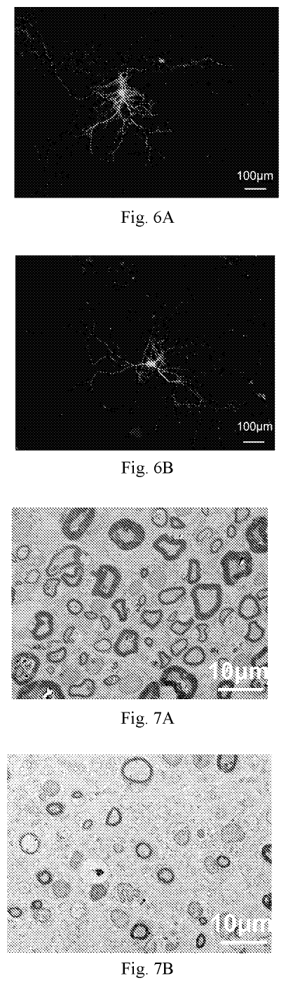

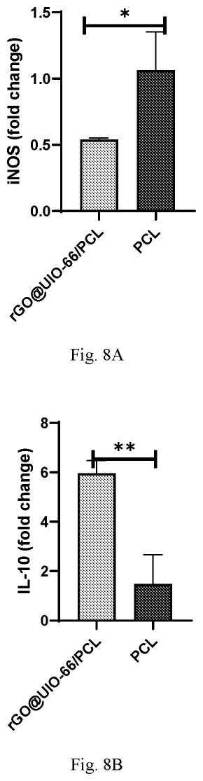

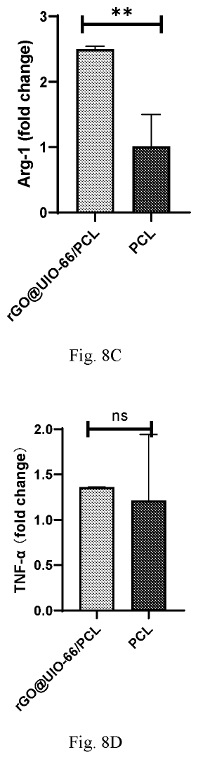

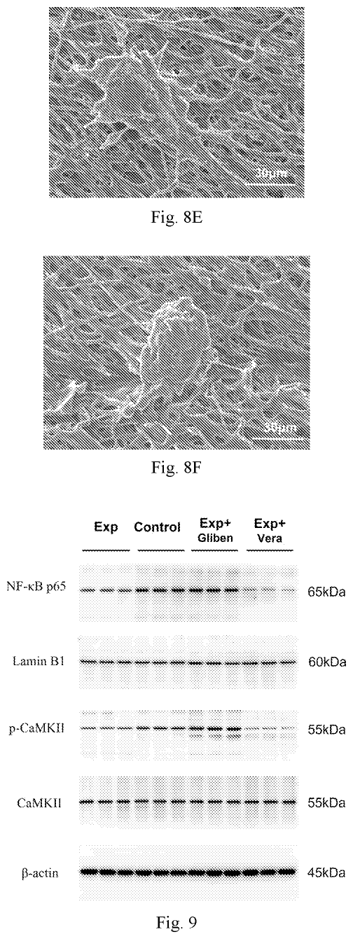

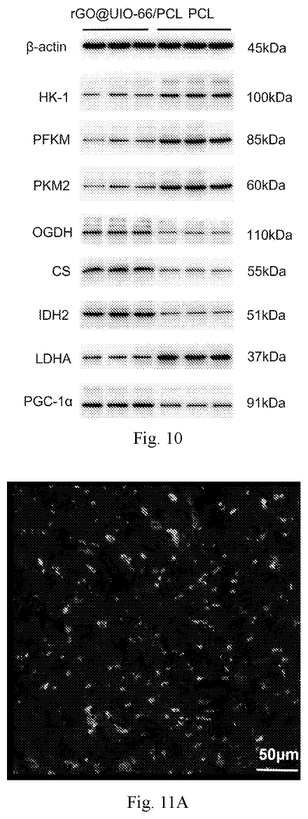

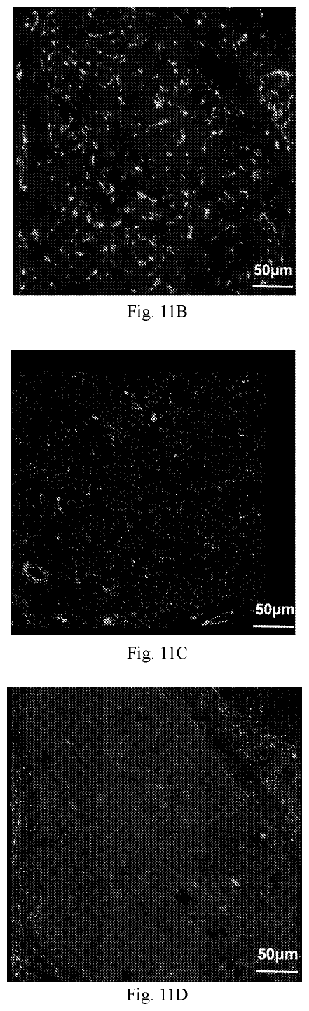

The piezoelectric conductive composite stent prepared by the example and the PCL catheter stent prepared by the control example were carried out in vitro experiments and in vivo experiments respectively, and the results are shown in FIGS. 6-11: the piezoelectric conductive composite stent of the present invention can affect the bioelectricity level of macrophage cell membranes, reduce calcium ion influx and thereby reduce the expression of inflammatory signaling pathways, and at the same time promote utilization and productivity of glucose in cells by changing the macrophage metabolic mode, thereby regulating and controlling the polarization of macrophages from proinflammatory phenotype to anti-inflammatory phenotype. The piezoelectric conductive composite stent of the present invention can promote macrophage infiltration in the early stage of nerve injury, and promote the transformation of macrophages from proinflammatory to anti-inflammatory phenotype in the late stage of nerve repair.

### Application Example

The piezoelectric conductive composite stent prepared by the example was implanted into the nerve defects in the animal, and non-invasive ultrasonic physiotherapy was performed on the surface of the site where the piezoelectric conductive composite stent is embedded with the help of a handheld ultrasound machine (Primo therasonic 460, EMS physio, UK) for 10-30 minutes every day at the frequency of 1 MHz. The strength is 1.5 W/cm², which stimulates the piezoelectric effect of the stent.

In summary, the present invention constructs a piezoelectric conductive composite stent by coaxial electrostatic spinning integrated forming technology, using nanoparticles of UIO-66-NH2 to act as piezoelectric catalytic response materials, using nanoparticles of graphene or its derivatives to act as conductive materials, under the mechanical force of ultrasonic waves, the piezoelectric crystals in the stent are deformed, and the self-generated electricity is conducted by conductive particles, promoting the transmission of electrical signals on the surface of the stent from the proximal end to the distal end, and guiding the elongation of the distal end of the regenerative nerve axon. The material has small toxic, side effects, and good biocompatibility, implantation of an external power supply or electrode is not needed, the tissue regeneration speed can be effectively improved, the pain and inconvenience of patients is reduced, and the risk of infection is decreased. The piezoelectric conductive composite stent of the present invention can affect the bioelectricity level of macrophage cell membrane, reduce calcium ion influx and thereby reduce the expression of inflammatory signaling pathway, and at the same time promote utilization and productivity of glucose in cells by changing the macrophage metabolic mode, thereby regulating and controlling the polarization of macrophages from proinflammatory phenotype to anti-inflammatory phenotype. The piezoelectric conductive composite stent of the present invention combines with ultrasound-assisted therapy, therefore, it can promote axon regeneration and myelination, and has a good clinical application prospect.

The above is only a better embodiment of the present invention, and does not therefore limit the embodiment and scope of protection of the present invention, and those skilled in the art should be able to realize that all solutions obtained by equivalent substitution and obvious changes made by the description and illustration of the present invention should be included in the scope of protection of the present invention.

What is claimed is:

1. A piezoelectric conductive composite stent, wherein a length of the piezoelectric conductive composite stent is 1 cm-3 cm, an inner diameter of the piezoelectric conductive composite stent is 2.5 mm-3.5 mm, and a thickness of a pipe wall of the piezoelectric conductive composite stent is 0.4 mm-0.45 mm; the piezoelectric conductive composite stent comprises: an inner layer and an outer layer sleeved outside the inner layer, and a plurality of nano grooves are provided in a peripheral face of the outer layer;
wherein the inner layer is prepared from polycaprolactone dissolved in a binary organic solvent;
the outer layer is prepared from polycaprolactone dissolved in a binary organic solvent, polyvinylpyrrolidone dissolved in a binary organic solvent, nanoparticles of metal organic framework materials, and nanoparticles of graphene or its derivatives, the metal organic framework material is UIO-66-NH₂.

2. The piezoelectric conductive composite stent according to claim 1, wherein the graphene or its derivatives comprise: at least one of graphene, graphene oxide or reduced graphene oxide.

3. The piezoelectric conductive composite stent according to claim 1, wherein the binary organic solvent is a dichloromethane/dimethylformamide organic solvent, and the volume ratio of dichloromethane to the dimethylformamide is in the range of 2 to 4.

4. A method for preparing the piezoelectric conductive composite stent according to claim 1, comprises the steps of:
S1, adding polycaprolactone to a binary organic solvent, and after sonication, obtaining a spinning liquid of the inner layer; adding polycaprolactone and polyvinylpyrrolidone to binary organic solvents, and after sonication, adding nanoparticles of graphene or its derivatives and nanoparticles of metal organic framework materials, and after uniform treatment, obtaining a spinning liquid of the outer layer;
S2, electrostatic spinning the spinning liquid of the inner layer prepared by step S1 and the spinning liquid of the outer layer prepared by step S1 together, and after drying, washing several times, obtaining the piezoelectric conductive composite stent.

5. The preparation method according to claim 4, wherein in step S1, the temperature of the sonication is 10° C.-20° C., and the time of the sonication is 20 min-40 min.

6. The preparation method according to claim 4, wherein in step S1, the mass concentration of polycaprolactone in the spinning liquid of the inner layer is 15%-20%.

7. The preparation method according to claim 4, wherein in step S1, in the spinning liquid of the outer layer, the mass concentration of polycaprolactone is 8%-12%; the mass concentration of polyvinylpyrrolidone is 4%-8%; the mass concentration of nanoparticles of graphene or its derivatives is 1%-2%; the mass concentration of nanoparticles in metal organic framework materials is 1%-2%.

8. The preparation method according to claim 4, wherein in step S2, the electrostatic spinning comprises: adding, respectively, the spinning liquid of the inner layer and the spinning liquid of the outer layer to two syringes, wherein the two syringes share a nozzle, a model number of the nozzle is 19, a voltage of the spinning is 10 kV-20 kV, a receiving distance of the receiving rod is 18 cm-20 cm, and a speed of the push pump is 1.8 mL/h-2.5 mL/h; the mold speed for electrostatic spinning the inner layer is 10 rpm-20 rpm, and the mold speed for electrostatic spinning the outer layer is 70 rpm-90 rpm for the outer layer of electrospinning.

9. The preparation method according to claim 4, wherein in step S2, the detergent used for washing comprises: alcohol or/and water.
